(19) United States
(12) Patent Application Publication
(10) Pub. No.: US 2024/0157358 A1
(43) Pub. Date: May 16, 2024

(54) BIOSENSING SYSTEM AND METHOD FOR IN-SITU DETERMINATION OF METASTASIS IN A SAMPLE

(71) Applicant: City University of Hong Kong, Hong Kong (CN)

(72) Inventors: Bee Luan KHOO, Singapore (SG); Jing ZHANG, Hong Kong (CN); Wei LI, Hong Kong (CN); Yatian FU, Hong Kong (CN)

(21) Appl. No.: 18/494,108

(22) Filed: Oct. 25, 2023

Related U.S. Application Data
(60) Provisional application No. 63/383,516, filed on Nov. 14, 2022.

(51) Int. Cl. B01L 3/00; G01N 21/65

(52) U.S. Cl. CPC B01L 3/502715; B01L 3/502707; G01N 21/658; B01L 2300/0636

(57) ABSTRACT

The present invention provides a biosensing system including a microfluidic-based biosensor designed for both point-of-care testing and a home-based assessment. A method for distinguishing a metastatic sample from a non-metastatic sample in-situ based on an observation of relative distribution of organisms in a biosensor of the present system in response to different test samples through a portable imaging device or an imaging module integrated into a smartphone or mobile device is also provided. Chemotactic preference of the organisms to a sample is quantified by using Raman spectroscopy to generate a profile of target analytes for determination of metastatic status and potential in a particular sample.

### CROSS-REFERENCE TO RELATED APPLICATION

This application claims priority from the U.S. provisional patent application Ser. No. 63/383,516 filed Nov. 14, 2022, the disclosure of which is incorporated herein by reference in its entirety.

### TECHNICAL FIELD

The present invention generally relates to an ultrasensitive, label-free biosensing system for determining metastatic status of a sample in-situ, in particular, to a biosensing system for determining the metastatic status of liquid biopsies from a subject susceptible to or suffering from cancer suitable for both point-of-care testing and personalized medicine.

### BACKGROUND

The following references are cited and discussed herein:
1. Buus, T.W., et al. (1), Comparison of contrast-enhanced CT, dual-layer detector spectral CT, and whole-body MRI in suspected metastatic breast cancer: a prospective diagnostic accuracy study. Eur Radiol, 2021. 31(12): p. 8838-8849.
2. Buus, T.W., et al. (2), Breast cancer: comparison of quantitative dual-layer spectral CT and axillary ultrasonography for preoperative diagnosis of metastatic axillary lymph nodes. Eur Radiol Exp, 2021. 5(1): p. 16.
3. Christensen, E., et al., Early Detection of Metastatic Relapse and Monitoring of Therapeutic Efficacy by Ultra-Deep Sequencing of Plasma Cell-Free DNA in Patients With Urothelial Bladder Carcinoma. J Clin Oncol, 2019. 37(18): p. 1547-1557.
4. Coombes, R.C., et al., Personalized Detection of Circulating Tumor DNA Antedates Breast Cancer Metastatic Recurrence. Clin Cancer Res, 2019. 25(14): p. 4255-4263. Inaba, S., et al., Accuracy evaluation of the C. elegans cancer test (N-NOSE) using a new combined method. Cancer Treat Res Commun, 2021. 27: p. 100370.
5. Khoo, B.L., et al., Detection of Clinical Mesenchymal Cancer Cells from Bladder Wash Urine for Real-Time Detection and Prognosis. Cancers (Basel), 2019. 11(9). Kusumoto, H., et al., Efficiency of Gastrointestinal Cancer Detection by Nematode-NOSE (N-NOSE). In Vivo, 2020. 34(1): p. 73-80.
6. Parsons, H.A., et al., Sensitive Detection of Minimal Residual Disease in Patients Treated for Early-Stage Breast Cancer. Clin Cancer Res, 2020. 26(11): p. 2556-2564.
7. Wang, X., et al., Stimuli-Responsive Nanodiamond-Based Biosensor for Enhanced Metastatic Tumor Site Detection. SLAS Technol, 2018. 23(1): p. 44-56.
8. Zhang, J., et al., Worm-based microfluidic biosensor for real-time assessment of the metastatic status. Cancers, 2021. 13(4): p. 873.
9. Zhou, Z., et al., MRI detection of breast cancer micrometastases with a fibronectin-targeting contrast agent. Nat Commun, 2015. 6: p. 7984.

Metastasis is a process of cancer spreading to other organs in which primary tumor cells migrate and establish secondary tumors by invading specific tissues or disseminating blood and lymphatic systems. Patients who develop metastases often have a poor prognosis, posing a great challenge to cancer treatment. Early detection of metastatic disease is of great significance for reducing mortality and improving overall survival to facilitate timely treatment and intervention. It is crucial to provide patients with timely and effective anticancer treatment in the early stages of metastasis. However, current treatments for most cancer patients are often delayed due to the complicated process required for conventional diagnostic assays.

Breast cancer is one of the world's most malignant and deadly cancers and has surpassed lung cancer as the most commonly diagnosed cancer. The occurrence of metastases is highly associated with increased organ tropism and mortality. However, early identification of metastatic status in breast cancer remains challenging due to the lack of sensitive biomarkers and cost-effective approaches.

Conventional techniques to assess the metastatic status of the most common tumors, e.g., breast tumors, include MRI and CT (some other conventional techniques are summarized in Table 1). However, these gold standards often require large and highly specialized equipment. Extraction of circulating tumor cells (CTCs), circulating tumor DNA (ctDNA), and other cells from blood samples for histological analysis is also moderately invasive and can be time-consuming. Furthermore, the heterogeneity of reads leads to the need for extensive downstream processing and evaluation by trained personnel.

Therefore, point-of-care biosensors with high sensitivity, low fabrication and operational cost to rapidly assess tumor metastatic status are pivotal for clinical tumor diagnosis and routine treatment evaluation. In addition, since Covid-19 pandemic, there has been an increasing demand for personalized medicine including test kits and rapid detection platforms for certain categories of chronic diseases or conditions. Non-acute patients tend to choose not to attend healthcare centers, clinics, or hospitals to have routine diagnostic tests or medical check-ups but a more personalized approach such as home-use diagnostic kits to check their health or even tumor metastatic status regularly if they can handle the testing by themselves. Healthy individuals who intend to have more specific medical check-ups for certain pathologies at the hospital or medical center may also wish to have some relatively simple diagnostic tests be done in advance at home such as urine or blood tests in order to minimize the duration of stay at the healthcare centers, clinics or hospitals. With an aid of other advanced technologies such as miniaturized imaging devices and electronics being integrated into a smartphone or other mobile devices, individuals can easily capture high quality images of the test results obtained from portable biosensors or test kits and share the image data with the respective medical professionals, pathologists or practitioners in a secure platform.

Hence, an easy-to-use platform that is preferably label-free, ultrasensitive, and designed for both point-of-care testing and personalized testing to monitor health and metastatic status of an individual and capable of generating test data that can be captured and transferred by the individual to a respective medical professional, pathologist, or practitioner without special skills or training, and even the data can be instantly assessed optically by the user through ordinary portable electronic devices, is an unmet need.

### SUMMARY OF INVENTION

Accordingly, a first aspect of the present invention provides a biosensing system including a microfluidics-based biosensor, where the microfluidics includes multiple layers including:
a first layer including at least a plurality of fluid inlets for loading one or more samples and a window; and
a second layer disposed under the first layer and including an organism loading region and a plurality of bio-incubation chambers each having a plurality of obstructing mechanisms so that a type of organisms incubated therein is selectively attracted or repelled by one or more analytes in order to move away from the bio-incubation chambers towards the one or more analytes or stay within the bio-incubation chambers,
wherein a presence of the one or more analytes in the one or more samples indicates a metastatic status of the samples or a subject from which the samples are obtained.

In certain embodiments, each of the plurality of fluid inlets is disposed adjacent to an edge of the window.

In certain embodiments, the window is disposed substantially at a center of the first layer.

In certain embodiments, each of the plurality of bio-incubation chambers is arranged in fan-like shape diverging substantially from a center of the second layer from a plan view of the biosensor.

In certain embodiments, the obstructing mechanisms in each of the plurality of bio-incubation chambers are a plurality of polygonal structures.

In certain embodiments, the obstructing mechanisms are a plurality of triangular prism-shaped structures.

In certain embodiments, each of the triangular prism-shaped structures has a triangular top surface with an average length of 2 mm on each side.

In certain embodiments, each of the plurality of polygonal structures is evenly spaced apart from the other to define a plurality of runways for the type of organisms to move away from the bio-incubation chambers towards the one or more analytes.

In certain embodiments, there are at least two fan-like shaped bio-incubation chambers arranged on the second layer.

In certain embodiments, the organism loading region is disposed at an intersection of different bio-incubation chambers.

In certain embodiments, each of the plurality of bio-incubation chambers has a cavity to accommodate the type of organisms and allow for a culture medium to incubate with the organisms.

In certain embodiments, the type of organisms is a kind of nematodes.

In certain embodiments, the type of organisms is Caenorhabditis elegans (C. elegans).

In certain embodiments, the culture medium is nematode growth medium (NGM).

In certain embodiments, the first layer communicates with the second layer through one or more fluid channels, where each of the fluid channels or each intersection of two or more of the fluid channels has a larger width but a smaller height from their cross-section than those of fluid channels within the bio-incubation chambers.

In certain embodiments, each of the fluid channels from the first layer until reaching the bio-incubation chambers of the second layer has an average width of about 500 μm, each intersection of two or more of the fluid channels has an average width of at least 2 mm, and both the fluid channels and the intersection of the fluid channels have an average height of about 1 mm, while each of the fluid channels within the bio-incubation chambers has an average width of about 8 mm and an average height of about 5 mm, where the average width of the fluid channels within the bio-incubation chambers is also an average diameter of the bio-incubation chambers.

In certain embodiments, the samples loaded into the plurality of fluid inlets can have a volume of smaller than 1 mL at each loading.

In certain embodiments, seven different samples can be tested by the present biosensing system at once.

In certain embodiments, the window of the first layer is configured to allow a user of the biosensing system to observe any movement of the organisms from the organism loading region where they are loaded toward a direction where the samples are transported from the first layer to the second layer.

In certain embodiments, the samples can be biological fluids, body fluids, metabolites, extracts from tissues or lesions, or media containing any of the above.

In certain embodiments, the samples are liquid biopsies including, but not limited to, urine, saliva, mucus secretion, and extracts from tissues or lesions.

In certain embodiments, the subject is human or non-human animal.

In certain embodiments, the one or more analytes include urea, urine-derived metabolites including L-proline, glucose, trisodium citrate, lactic acid, glutamic acid, CaCl₂, MgSO₄, KPO₄, citric acid, glutamine, gamma-aminobutyric acid, and L-pyroglutamic acid, and biological cells including circulating tumor cells, tumor-initiating cells, and cancer stem cells.

In certain embodiments, the analytes can be chemoattractant or chemorepellent to the type of organisms.

In certain embodiments, the one or more analytes are glutamate, urea and L-proline.

In certain embodiments, the cancer cells of a malignant phenotype include breast cancer cells and potentially any cancer type with a corresponding analyte that can be detected.

Optionally, the biosensing system further includes a holder of the microfluidics, a linear translation stage, and a lens disposed under the microfluidics, where the holder can be attached to the linear translation stage for securing the microfluidics on the linear translation stage.

In certain embodiments, the linear translation stage is a precision z-axis translation stage with fixed x- and y-axis positions.

The lens can be a droplet lens or microlens for use to magnify an area of interest in images of the microfluidics captured by a smartphone or mobile device integrated with an imaging module. Alternatively, the lens may be substituted with an imaging device configured to capture magnified images, where the imaging device is capable of outputting the corresponding image data for subsequent analysis.

A second aspect of the present invention provides a method for differentiating a metastatic sample from a non-metastatic sample including providing the samples to the biosensing system described in the first aspect, where the biosensor of the biosensing system is loaded with a type of organisms that will be attracted or repelled by one or more analytes in the metastatic sample.

In certain embodiments, a standard derived from a non-metastatic cell line medium, a control sample with a known concentration of one or more analytes, and a blank sample (without any analytes or cells) are also loaded into the biosensor.

The method in the second aspect also includes observing any movement of the organisms from where they are loaded towards a direction where the samples are loaded from the first layer and transported to the second layer of the biosensing system and quantifying the movement in terms of the percentage of the moving organisms towards where the samples are loaded and transported relative to the percentage of the moving organisms towards where the standard is loaded and transported in order to determine a chemotaxis index (CI) of the organisms to a particular sample relative to the percentage of the moving organisms towards the standard.

In certain embodiments, what if the CI is greater than 2, the sample is considered metastatic; otherwise, the sample is considered non-metastatic if the CI is between 1 and 2.

In certain embodiments, the method further includes generating a Raman intensity profile for one or more of the analytes from the control sample in terms of Raman intensities measured at one or more peaks distinctive to the one or more analytes and comparing the Raman spectrum of the test sample with the Raman intensity profile generated from the control sample in order to validate the metastatic status of the test sample.

A third aspect of the present invention provides a kit for determining metastasis of a biological sample including the biosensing system described in the first aspect and optionally one or more additional components according to certain embodiments including the microfluidics holder, precision z-axis translation stage, and/or the droplet lens for capturing images of the microfluidics and observing the test results in-situ by a smartphone or mobile device integrated with an imaging module or by an imaging device.

In certain embodiments, the microfluidics is a microfluidic chip.

In certain embodiments, the kit also includes a plurality of containers for carrying and loading test samples, organisms, a control sample, and a standard into the biosensor of the biosensing system, respectively.

Optionally, one or more of the plurality of containers may be substituted with a liquid handling means such as a syringe for loading the samples, organisms, control and standard into different parts of the biosensing system.

In certain embodiments, the test samples, the control sample and the standard are loaded into any three of the fluid inlets of the first layer.

In certain embodiments, the organisms are loaded into the organism loading region of the second layer which is substantially disposed at about a center of the second layer from a plan view.

In certain embodiments, the precision z-axis translation stage includes a stage shaft, a rotor, a plurality of clamps and screws.

Other aspects of the present invention include how to fabricate the biosensing system described in the first aspect or according to certain embodiments described herein and how the biosensing system may be optimized for determining different disease or cancer stages of other cancer cell types.

In the aspect relating to a method of fabricating the biosensing system, the method includes providing different molds for different layers of the microfluidics, injection molding one or more thermoplastics into the corresponding molds to form templates of different layers, patterning on the templates to form corresponding fluid channels and/or cavities in accordance with the design or system requirements for each biosensing assay, and bonding different layers by any feasible physical or chemical process. Alternatively, other techniques that are capable to form multilayered microfluidics such as 3-D printing can be used. Materials for forming the microfluidics of the present biosensing system can be any materials suitable for injection molding, lithography, and/or 3-D printing, including but not limited to, polydimethylsiloxane (PDMS), a mixture or co-polymer comprising the same. In the embodiments where nematodes are used as the type of organisms for biosensing an analyte abundant in metastatic cancer samples, the chambers for incubating the nematodes with the corresponding culture medium are configured to have a plurality of obstructing mechanisms such that the nematodes are required to go through a non-straight runway from where they are loaded and incubated toward the metastatic cancer sample.

This summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. Other aspects of the present invention are disclosed as illustrated by the embodiments hereinafter.

### BRIEF DESCRIPTION OF DRAWINGS

The appended drawings, where like reference numerals refer to identical or functionally similar elements, contain figures of certain embodiments to further illustrate and clarify the above and other aspects, advantages and features of the present invention. It will be appreciated that these drawings depict embodiments of the invention and are not intended to limit its scope. The invention will be described and explained with additional specificity and detail through the use of the accompanying drawings in which:

FIG. 1A shows a structure of a conventional worm-based (WB) biosensor.

FIG. 1B schematically depicts a structure of the present biosensing system according to certain embodiments, where it shows a perspective view of the biosensing system (left), an exploded view of a two-layered microfluidics (right), and an inset showing partially a bio-incubation chamber on one of the layers of the microfluidics.

FIG. 2 shows detection results of metastatic and non-metastatic phenotypes by the present biosensing system: (A) Box plot reflecting the chemotactic preference of C. elegans with the biosensor for less metastatic cancer samples (MCF-7), and healthy controls (fibroblast). C. elegans counts were normalized to those obtained with PBS buffer standards; (B) Bar chart reflecting the chemotactic preference of worms to samples from more metastatic cancer cells (MDA-MB-231) compared to samples from less metastatic cancer cells (MCF-7). The dotted line corresponded to the proportion of C. elegans with samples from healthy controls (fibroblast); (C) Proportion of C. elegans when exposed to samples denatured by heat treatment; (D) Proportion of C. elegans when exposed to samples obtained from larger clusters of higher metastatic potential, where the dotted line corresponds to the proportion of C. elegans with samples from PBS buffer standards; *** states for p<0.0001, ** states for p<0.005. * states for p values <0.05.

FIG. 3 shows chemotactic response to glutamate by metastatic and non-metastatic phenotypes in the present biosensing system: (A) Glutamate levels in samples from cancer cells of the more metastatic phenotype (MDA-MB-231) and cancer cells of the less metastatic phenotype (MCF-7); (B) Quantitative evaluation of outcomes from the present biosensor by establishing a CI. A range of glutamate concentrations (0.02 mg/ml, 0.04 mg/ml, 0.06 mg/ml, 0.08 mg/ml, and 0.1 mg/ml) was evaluated; (C) Proportion of C. elegans under exposure to samples from cancer cells of the more metastatic phenotype (MDA-MB-231) and cancer cells of the less metastatic phenotype (MCF-7) before and after glutamate degradation. The dotted line corresponded to the averaged value obtained with PBS buffer standards; (D) Evaluation of the detection limit for the present biosensor, where the dotted line (1) reflects the threshold determined by the rate of false-positives (8.71±0.033%); the dotted line (2) reflects the threshold determined by the averaged value obtained with undiluted MCF-7 samples (25.73±0.054%); *** states for p<0.0005, **states for p<0.005 * states for p<0.05.

FIG. 4A shows microscopic images of nematodes in the present biosensing system treated with metastatic urine sample, MCF-7 standard (non-metastatic cancer control), and PBS only (negative control) (upper panel); and the difference in chemotactic preference of nematodes to metastatic urine sample compared with MCF-7 standard in terms of chemotaxis index (CI) (lower panel) relative to the CI of PBS only (CI_{PBS}), where **** indicates that p-value less than 0.00005.

FIG. 4B shows microscopic images of nematodes (C. elegans) in the present biosensing system treated with non-metastatic urine sample, MCF-7 standard, and PBS only (upper panel); and the chemotactic difference between non-metastatic urine sample and MCF-7 standard in terms of their CI_{PBS}.

FIG. 4C shows microscopic images of nematodes in the present biosensing system treated with healthy individual urine sample, MCF-7 standard, and PBS only (upper panel); and the chemotactic difference between healthy individual urine sample and MCF-7 standard in terms of their CI_{PBS}, where * indicates that p-value less than 0.05.

FIG. 4D shows a box plot of chemotactic preference of nematodes to metastatic, non-metastatic, and healthy individual urine samples in terms of their CIs obtained from FIGS. 4A-4C, respectively, relative to the CI towards MCF-7 standard (CI_{MCF-7}), where **** indicates that p-value less than 0.00005; *** indicates that p-value less than 0.0005

FIG. 4E shows distributions of metastatic urine sample, non-metastatic urine sample, and healthy individual urine sample in terms of their CI_{MCF-7}.

FIG. 5A shows a quantitative assessment of the chemotactic preference of nematodes in terms of CI² measured and calculated according to certain embodiments of the present invention towards single metastatic cancer cells (MDA-MB-231), a mixture of metastatic cancer cells and white blood cells (MDA-MB-231 in WBCs), white blood cells (WBC) and PBS only (negative control).

FIG. 5B shows a quantitative assessment of the chemotactic preference of nematodes in terms of CI² measured and calculated according to certain embodiments of the present invention towards an increasing number of single metastatic cancer cells (1, 10, 100, and 1000) with respect to WBC and PBS only.

FIG. 5C shows a quantitative assessment of the chemotactic preference of nematodes in terms of CI² measured and calculated according to certain embodiments of the present invention towards an increasing number of single metastatic cancer cells (1, 10, 100, and 1000) mixed with WBC compared to WBCs and PBS only.

FIG. 6 shows an analysis of Raman spectrum profiles of clinical urine samples, urea, and L-proline: (A) the Raman spectrum of urine samples from breast cancer patients with and without metastasis, in which four distinct differences were observed at 856, 914, 1008, and 1048 cm⁻¹ corresponding to urea and L-proline; (B) the baseline corrected Raman spectrum of urea with a baseline of 40 mg/mL urea solution, where one significant prominent peak was observed at 1008 cm⁻¹; (C) the baseline corrected Raman spectrum of L-proline with a baseline of 1000 mg/mL L-proline solution, where there were three prominent peaks observed at 856, 914, and 1048 cm⁻¹; (D) and (E) show calibration curves of Raman intensities measured at different concentrations of urea (40, 20, 10, 5 and 2.5 mg/mL) and L-proline (1000, 500, 250, 125, and 72.5 mg/mL) baseline solutions, respectively.

FIG. 7 shows urea and L-proline profiles in different urine samples: (A) Concentration of urea in metastatic and non-metastatic samples of cancer patients; (B) Concentration of L-proline in metastatic and non-metastatic samples of cancer patients; (C) Heat map reflects nematodes' preference for various urea concentrations; (D) Heat map reflects nematodes' preference for various L-proline concentrations; (E) Two-dimensional dot plot reflecting the correlation between CI_{MCF-7} and urea concentration of cancer patients; (F) Two-dimensional dot plot reflecting the correlation between CI_{MCF-7} and L-proline concentration of cancer patients, where **** indicates that the p-value is less than 0.00005; M: metastatic samples; NM: non-metastatic samples.

FIG. 8 shows Raman intensity of (A) urea and (B) L-proline from urine samples of cancer patients (M: metastasis; NM: non-metastasis), where **** indicates that the p-value is <0.00005;

FIG. 9 shows a heat map reflecting nematodes' preference for level of urea and L-proline in clinical samples in terms of average urea and L-proline concentration values in metastatic and non-metastatic samples, where M: 3.4 mg/ml urea+0.14 g/ml L-proline; NM: 7.5 mg/mL urea+0.32 g/ml.

FIG. 10 schematically depicts certain embodiments of the present biosensing system integrated with optional components for an alternative image data capturing and processing through a camera of a smartphone by a user and workflow of using the same for sample handling, qualitative and quantitative assessments of the results in a point-of-care setting: (A) a schematic diagram of the biosensing system including the biosensor (microfluidic chip), Z stage with chip holder, and a droplet lens (in the inset) for use with a smartphone; (B) representative microscopy images of the assay results from a blank chamber (upper image) and a chamber with nematodes (lower image), where the scale bar is 1 mm; (C) a proposed workflow from sample collection, data capturing, processing and analysis in the point-of-care setting, in which step [01]: Collect the clinical sample using a collection tube; step [02]: Return the sample to the lab within 8 hours of collection; step [03]: Assay operations; step [04]: Data analysis; step [05]: CI index evaluation.

FIG. 11 schematically depicts another proposed workflow from sample collection, data capturing, processing and analysis in a remote setting (e.g., user's home) based on a kit comprising the biosensing system depicted in FIG. 10A, in which step [01]: Set-up of the kit; step [02]: Collect the sample using a collection tube; step [03]: Assay operations and evaluation with integrated components, where tubes labeled C, P, U, and S contain nematodes, PBS, urine samples collected in step [01], and standards, respectively, and tubes P, U and S are added to the three inlets of the first layer and tube C is added to the center inlet of the second layer; step [05]: CI index evaluation through a smartphone or upload images for automated analysis.

FIG. 12 shows nematodes' viability, images, chemotactic preference to MCF-7 samples and temperature change over time in a storage component according to the embodiments of the biosensing system depicted in FIG. 10A and the workflow depicted in FIG. 11: (A) C. elegans viability after 6 h, 8 h and 24 h in the 1.5-ml tube; (B) Images of C. elegans after 24 h in the 1.5-ml tube; (C) CI_{MCF-7} of metastatic cancer tested by C. elegans in the 1.5-ml tube after 24 h, where CI_{MCF-7}>2 reflects the presence of metastasis; (D) Temperature change of samples stored in an ice box over time.

FIG. 13 shows nematodes' viability in the embodiments of the biosensing system depicted FIG. 10A and operational flow rate used therein: (A) Viability of C. elegans in the biosensor compared to those in an agar plate; (B) Time required for using different flow rates (0.25, 0.5, 1, and 1.5 m1/min) of pressure pump to flow samples.

FIG. 14 shows a design and a prototype of the z stage according to certain embodiments: (A) Schematic diagram of the z stage from a perspective view; (B) Representative photo from a perspective view of a prototype of the z stage with a dimension of 40.28 (L)×40 (W)×13 mm (H), where a curved solid arrow represents a motion direction of the stage rotor, while a straight solid arrow represents a vertical motion direction of the stage shaft when the stage rotor moves in an anti-clockwise direction; (C) Another schematic diagram from a side view of the z stage; (D) Another representative photo from a side view of the prototype of the z stage.

Skilled artisans will appreciate that elements in the figures are illustrated for simplicity and clarity and have not necessarily been depicted to scale.

### DETAILED DESCRIPTION OF THE INVENTION

It will be apparent to those skilled in the art that modifications, including additions and/or substitutions, may be made without departing from the scope and spirit of the invention. Specific details may be omitted so as not to obscure the invention; however, the disclosure is written to enable one skilled in the art to practice the teachings herein without undue experimentation.

The present disclosure provides a biosensing system and a related kit useful for determining metastatic status of a sample in a label-free and non-invasive approach. The system requirements are relatively low, where only a microscopic device or even a portable imaging device, smartphone or mobile device integrated with a camera is sufficient to acquire the required data for subsequent analysis. The present biosensing system utilizes a chemotactic preference of a type of nematodes, Caenorhabditis elegans (C elegans), to one or more metabolites which are found to be more abundant in urine of metastatic breast cancer patients than urine from non-metastatic cancer patients or healthy individuals. C. elegans is a well-known model organism in behavioral dynamics due to its sensitive chemosensory properties. Response to chemical stimuli is a well-studied aspect of C. elegans behavior, but practical application thereof in assessing cancer metastasis is still not established yet. The present biosensing system together with the proposed method of evaluating the chemotactic preferences of C. elegans to various cancer samples provided hereinafter will validate its potential to develop into an accurate, specific, and sensitive biosensor in the area of cancer staging and prognosis.

Exemplarily, the biosensor of the present biosensing system is a multi-layered microfluidics. In certain embodiments, the biosensor is a two-layered microfluidic chip, in which a first layer comprises multiple fluid inlets to allow for injection of different test samples into the biosensor and a window to allow the user of the system for optical assessment of movements of nematodes in the biosensor. The window is disposed substantially at the center region of the first layer according to certain embodiments, where each of the fluid inlets is disposed adjacent to each edge of the window. The window can be in regular shape such as a rectangle or a circle, or irregular shape such as in a star-like or flower-like shape, so long as vision of the user towards the movements of the nematodes in the biosensor is not affected or blocked by the structure of the first layer.

In certain embodiments, a second layer of the biosensor comprises at least one fluid inlet for loading of the nematodes and a plurality of bio-incubation chambers for accommodating the nematodes. To facilitate optical assessment of the nematodes' movements responsive to different test samples, the at least one fluid inlet, or more specifically, the organism loading region, is preferably disposed substantially at the center of the second layer where different bio-incubation chambers intersect with each other. Before loading the organisms into the biosensor, the bio-incubation chambers are also loaded with a corresponding culture medium to the organisms. In each of the bio-incubation chambers, a plurality of obstacles is incorporated to provide one or more non-straight (or indirect) runways for the organisms to move from the point where they are loaded towards the direction where the test sample is loaded. Typically, each bio-incubation chamber communicates with one fluid inlet of the first layer through one or more fluid channels. Each of the plurality of obstacles has identical shape, dimension and distance from adjacent obstacles to each other according to certain embodiments in order to provide the runways with even width. The examples described hereinafter will provide more details as to the realization and enablement of the present biosensing system and using thereof for determination of metastatic status of a sample.

Table 1 below provides major differences between the present invention and some of conventional cancer staging diagnostic techniques in terms of the type of cancers to be analyzed, from what kind of sample, marker(s), sensing principle, duration of test, and possible sensitivity and specificity, etc.

TABLE 1

| | | | | | | Outcome | |
|---|---|---|---|---|---|---|---|
| | | | | | | (values in %: | |
| | | | | | | Sensitivity; | |
| | | | | | | Specificity, | |
| Method | Type | Sample | Marker(s) | Principle | Time | or description) | Ref. |
| Present | Breast | Urine | Metabolites | C. elegans | Within | 93.3%; | N/A |
| Invention | | | (e.g., glutamate, | sensing | hours | 88.2% | |
| | | | urea, L- | | | | |
| | | | proline, | | | | |
| | | | etc.); CTCs | | | | |
| Liquid | Bladder | Urine | exfoliated | Antibodies | Within | 90.9% ± 7%; no | Khoo |
| biopsy | | | bladder | | hours | specificity | et al. |
| | | | cancer cells | | | | (2019) |
| | Breast | Blood | ctDNA | Detecting | N/A | 89%; | Coombes |
| | | | | MRD | | 100% | et al. |
| | | | | | | | (2019) |
| | Bladder | Blood | ctDNA | | N/A | 100%; | Christensen |
| | | | | | | 98% | et al. |
| | | | | | | | (2019) |
| | Breast | Blood | cfDNA | | N/A | 81%; no | Parsons |
| | | | | | | specificity | et. al |
| | | | | | | provided | (2020) |
| CT | Breast | Tissue | Images | iodine | N/A | ~79.0%; | Buus |
| | | biopsy | | density, | | ~92.0% | et al. (2) |
| | | | | spectral | | | (2021) |
| | | | | slope, | | | |
| | | | | Z effective, | | | |
| | | | | conventional | | | |
| | | | | 1 CT HU | | | |
| | | | | values and | | | |
| | | | | ΔCE | | | |
| MRI | Breast | Human | Tissue | McNemar | N/A | ~65.0%; | Buus |
| | | Body | images | | | ~98.0% | et al. (1) |
| | | | | | | | (2021) |
| | Breast | Mice | fibrin- | FROC | N/A | Detection of | Zhou |
| | | | fibronectin | | | micrometastases | et al. |
| | | | complexes | | | of size >0.5 mm | (2015) |
| Fluorescence | HCC | Cell | MMP9 | Fluorescence | N/A | ND-MMP9 can | Wang |
| labelling | | lines | | intensity | | fluorescently | et al. |
| | | | | | | paint the | (2018) |
| | | | | | | surrounding | |
| | | | | | | area of | |
| | | | | | | metastatic | |
| | | | | | | cancer sites | |

### EXAMPLES

### Example 1

### Design, Structure and Fabrication of Biosensor

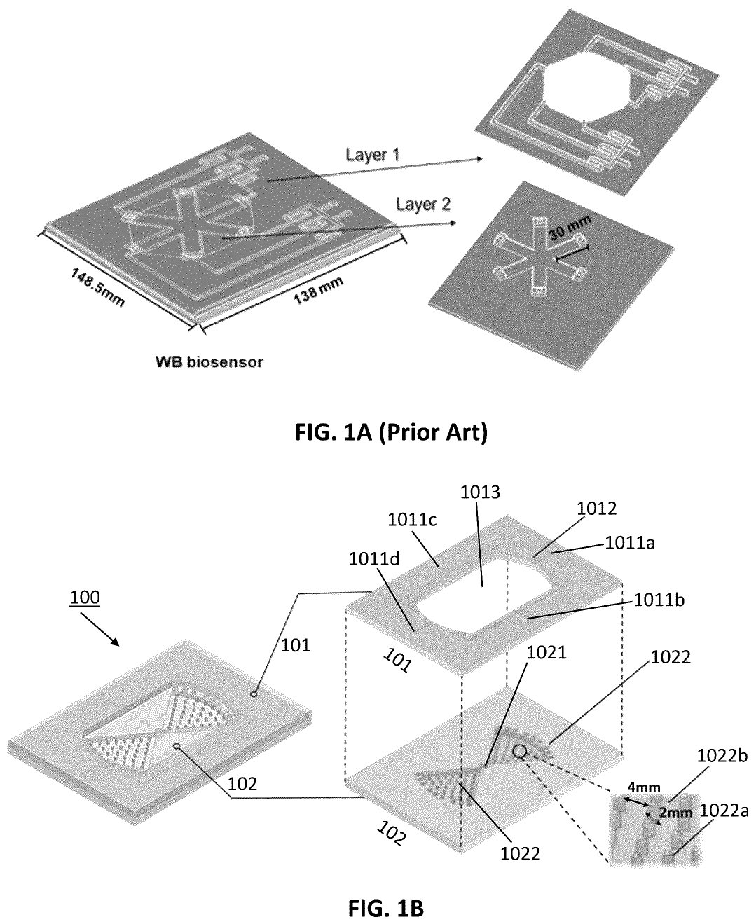

FIG. 1B provides an example of the present biosensing system, where the biosensor 100 comprises two layers (101, 102). The first layer 101 is disposed on top of the second layer 102. The first layer 101 comprises four fluid inlets (1011a, 1011b, 1011c, 1011d) and a plurality of fluid channels 1012 for fluid communication between the first layer 101 and the second layer 102. The first layer 101 also comprises a window 1013 for direct optical assessment (qualitative and quantitative) of any changes (e.g., movement of organisms from one end to the other) at the second layer 102. Optionally, the first layer may also be configured to have a closing mechanism (not shown in FIG. 1B) to close the window when needed such as after biosensing assay or during transportation of the biosensing system to avoid sample contamination.

At the second layer 102, two fan-like bio-incubation chambers 1022 which are substantially identical in size, shape and configuration. The two fan-like bio-incubation chambers 1022 intersect with each other at their respective head at which a loading region 1021 for loading the organisms for biosensing is disposed. In this example, although there are only two fan-like bio-incubation chambers 1022 provided on the second layer 102, it should be understood that more bio-incubation chambers can be provided if needed, for instance, if more samples (e.g., control, standard and comparative test sample) or dilutions of the same test sample need to be tested at the same time, or a higher throughput is required. In certain embodiments, the present biosensing system can screen up to 7 samples at each round of biosensing assay.

As seen from the inset showing a microscopic view of the configuration in one of the fan-like bio-incubation chambers 1022, the cavity of the chamber contains a plurality of triangular prism-like 3-D structures 1022a as obstacles each having a triangular top surface with a side length of about 2 mm each, where the obstacles are evenly distributed in said cavity such that each of them is spaced apart evenly from each other to create a number of runways 1022b with substantially identical width (each runway is ˜4 mm wide). In certain embodiments, the height of bio-incubation chambers 1022 (or the height of the runways 1022b) is larger than that of the inner wall of the fluid channels 1021 or the intersection of two or more fluid channels. For instance, the height of the bio-incubation chambers 1022 is about 5 mm while the height of the inner wall of the fluid channels 1021 or the intersection of two or more of them is only about 1 mm. The width of each of the fluid channels 1021 is about 500 μm and the intersection of two or more of the fluid channels has a width of about 2 mm. The height and the width of the fluid channels 1021 or the intersection of those fluid channels are not larger than the average diameter of the organisms such that only the fluid of the test samples can flow through from the first layer to the second layer while the organisms cannot go to the first layer through the fluid channels or their intersections.

In the biosensor of the present biosensing system, the provision of obstacles 1022a and runways 1022b in the fan-like bio-incubation chambers 1022 is intended to improve the accuracy of selection preferences of the organisms to different test samples during biosensing because the paths available from where the organisms are loaded towards the source of the test samples (i.e., the fluid channels 1022 connecting both the first and the second layers) become indirect (or not straight) in the presence of the obstacles 1022a. It should be understood that the configuration and distribution of the obstacles and runways in the bio-incubation chambers are not limited to those provided in this example, but can vary according to the needs, design of the biosensor, selection ability and behaviour of the organisms applied for biosensing, and their chemotactic preference, etc.

Typically, the biosensor was fabricated by standard lithography procedures. Premixed PDMS with 10% curing agent (SYLGARD™ 184 Silicone Elastomer Kit) was first poured into two different molds for the first and second layers of the biosensor, respectively, followed by curing overnight in 70° C. After curing, the two layers were aligned together manually or with an aid of computerized system and then sealed together through an oxygen plasma bonding technique. The corresponding fluid inlets, fluid channels, the organism loading region and bio-incubation chambers were patterned onto the respective layers before bonding. The sealed biosensor was sterilized by immersion in 70% ethanol and UV light for 30 min, followed by adding nematode growth medium (NGM) agar into the cavity of the bio-incubation chambers at the second layer until it was set.

C. elegans used in the present disclosure were obtained and cultured at The Hong Kong Polytechnic University. It was cultured in the OP50, a strain of Escherichia coli used to maintain C. elegans cultures, with nematode growth media (NGM) plate. Before each biosensing assay, the viability of C. elegans was assessed by probing with an inoculation needle.

All materials required for performing biosensing assay in the present biosensing system should be kept at room temperature before loading C. elegans into the biosensor to maintain their viability. Briefly, medium supernatants at −80° C. and biosensor initially stored in a 4° C. freezer were thawed at room temperature. Adult C. elegans were collected in 1.5 mL tubes and washed twice with phosphate-buffered saline (PBS) buffer (Gibco, Waltham, MA, USA) before assay. All clinical samples to be tested were transported at room temperature, and the biosensing assay should be completed within 4 hours from the time of collection. The clinical samples should be mixed well before loading, and about 1 ml of the sample was taken into a 1.5 ml tube for each biosensing assay. The remaining samples could be sealed and stored at −80° C. for further use.

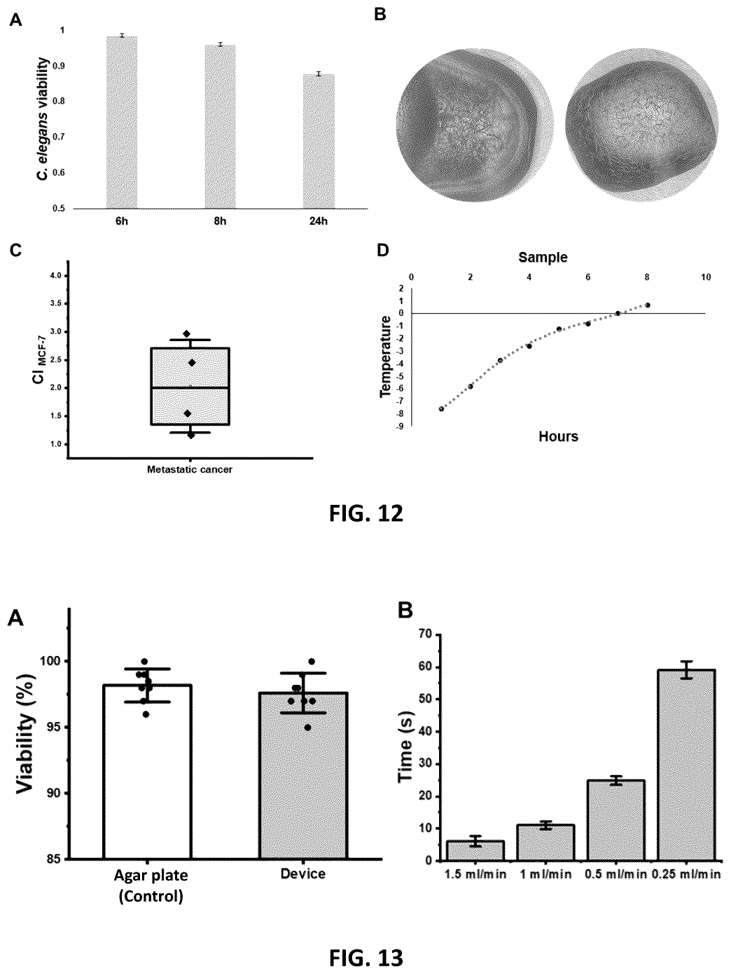

During each biosensing assay, approximately 50-100 μl of PBS containing 100 C. elegans was gently pipetted into the middle of the second layer where the organism loading region is disposed without compromising the viability of C. elegans and disrupting the integrity of the NGM agar, thereby increasing the reliability of the biosensing assay results. Once the C. elegans were settled, the pressure pump was started immediately to flow the test samples, comparative sample and/or negative control to the corresponding bio-incubation chambers from the fluid inlets at the first layer and through different fluid channels to reach the second layer without damaging the NGM agar integrity. In certain embodiments, an optimal flow rate of the fluid applied to the biosensor is about 0.5 mL/min. The difference in time duration required to have all fluid channels be primed with the fluid by applying different flow rates through a pump was evaluated and the results are shown in FIG. 13B. By this optimal flow rate, all fluid channels can be primed in 1 min and about 10 to 100 μl droplets can be formed at each fluid outlet of the fluid channels. Because of that, the volume of the test samples applied in each fluid inlet at each round of biosensing assay can be less than 1 mL. Normally, in the case of applying C. elegans as the organisms for biosensing, the nematodes started migration in 5 min after sample loading at this flow rate, and completed the distribution throughout the cavity of the bio-incubation chamber within about 40 min from when the samples were loaded. Inclusive of the assessment time, each biosensing assay by the present biosensing system using C. elegans as the organism model may take about 60 min or less.

In comparison, the worm-based (WB) biosensor by Zhang et al. (2021), a schematic briefly depicting the structure is shown in FIG. 1A, was much larger in size (138 mm×148.5 mm). The present biosensing system is also designed for on-chip observation and assessment in conjunction with a microlens magnifier attachable to a smartphone or mobile device integrated with rear-facing camera. The present biosensing system has more fluid channels to increase the number of samples to be handled at the same time compared to WB biosensor. The present biosensing system also does not have gradient generator to simplify the design and application of the test samples. Although a chemotaxis assay called Nematode-NOSE (or “N-Nose”) proposed by Inaba et al. (2021) and Kusumoto et al. (2020) enabled home sampling by using a 9-cm plate containing 10-mL NGM agar to accommodate nematodes, it did not have a miniaturized platform to enable portability and instantaneous optical assessment by the user at home before the assay plate reached the laboratory for testing. In contrast, the present biosensing system not just allows traditional phase contrast microscopy to analyze the result of the biosensing assay but also allows for instantaneous optical assessment by the user with a portable imaging device or smartphone/mobile device. The N-Nose was also not configured for multiplexing as it could only handle one sample at each instance. Table 2 below summarizes key differences between the present biosensing system, WB biosensor and N-Nose in terms of their functionalities:

TABLE 2

| Functionalities | Present Invention | WB Biosensor | N-Nose |
|---|---|---|---|
| Portable | Yes | No | Yes |
| Sample Multiplexing | Yes | Yes | No |
| Integrated on-chip | Yes | No | No |
| observation/assessment | | | |
| Home use | Yes | No | No |
| CTCs screening | Yes | No | No |
| potential | | | |

### Example 2

### Validation of Biosensing Ability in Terms of Chemotactic Difference Among Different Cancer Cells

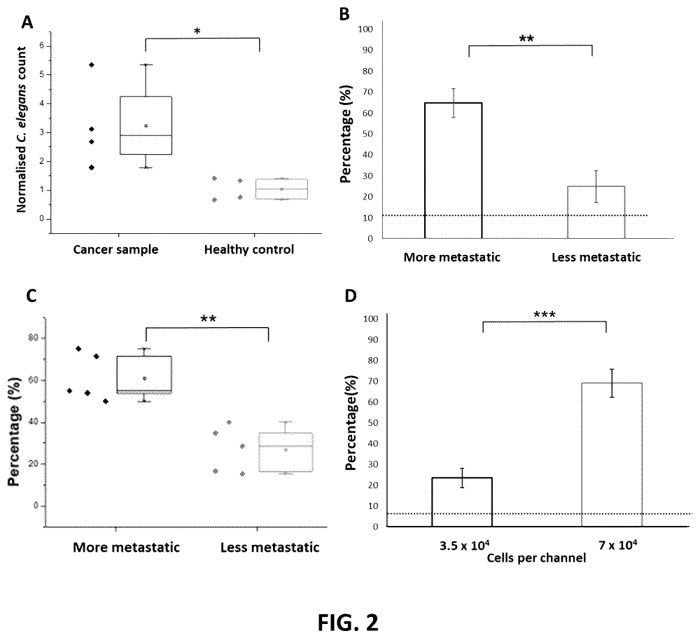

Cancer samples were distinguished from healthy controls by evaluating the chemotactic preference of C. elegans to samples obtained from cancer cell clusters with lower metastatic potential, using MCF-7 cell lines. FIG. 2A shows that the distribution of C. elegans was higher in channels loaded with samples from cancer clusters, compared to that from healthy fibroblast controls (3 folds, p-value <0.05).

The chemotactic preferences of C. elegans to samples from different cancer phenotypes were also investigated. Breast cancer cell lines MCF-7 and MDA-MB-231 represented the less metastatic and more metastatic cancer phenotypes, respectively. To standardize the quantitative readouts, a chemotaxis index (CI) relative to the CI measured from PBS (CI_{PBS}) was derived by the following Equation:

$${CI}_{PBS} = \frac{percentage of C . elegans attracted to sample 1}{percentage of C . elegans attracted to PBS} \qquad ( 1 )$$

The resultant CI_{PBS} level for samples obtained from cancer cell clusters with lower metastatic potential was still lower (3.24±1.52) compared to samples obtained from cancer cell clusters of higher metastatic potential (MDA-MB-231) (6.5±1.37; p<0.005). Specifically, compared with samples obtained from cancer cell clusters with lower metastatic potential (24.91±7.51%), C. elegans had a significantly higher chemotactic preference for samples obtained from cancer cell clusters of higher metastatic potential (64.72±6.98%, 2.6 folds) (FIG. 2B). These observations confirmed that cancer cells of higher metastatic potential provided different biochemical cues that could be detected by the present biosensor.

Interestingly, the denaturation of samples by heat treatment did not abolish the chemotactic preference of C. elegans to samples of higher metastatic potential. The persistence of chemotactic preference of C. elegans to metastatic samples suggested that the chemotactic agent for metastasis was not volatile or protein-based.

Clinically, larger tumors are associated with multifocal diseases. Compared with single-focal breast cancer, multifocal breast cancer has a higher risk of vascular invasion and lymph node metastasis. Larger tumor size is also associated with worsening patient prognosis. Therefore, to mimic the presence of larger tumors associated with a higher risk of metastatic disease, the cell seeding concentration was increased (7×10⁴ cells per channel) to produce larger cell clusters (increased by 4 folds; 8383.96±2373.2 μm²). FIG. 2D shows the preference of C. elegans for samples from larger cancer cell clusters (3 folds, 69.11%; p-value: 0.00003).

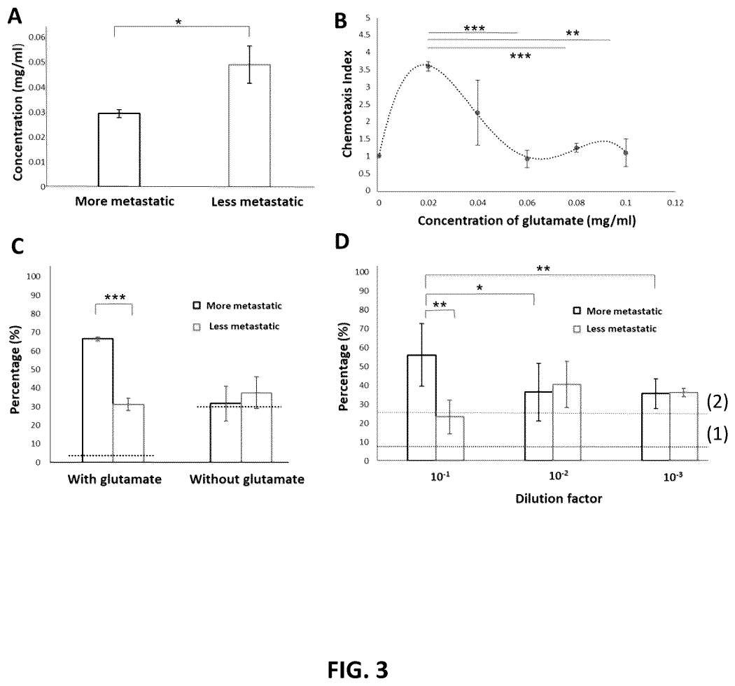

Glutamate is a component of glutamine metabolism in cancer cells associated with a malignant phenotype and is secreted by breast cancer cells at high concentration. Therefore, it is expected that glutamate could be a chemotactic agent for C. elegans to detect various cancer subtypes. Initially, the glutamate levels from samples of cancer cell clusters with various metastatic potential were quantified. FIG. 3A shows that the glutamate levels from samples of cancer cell clusters with less metastatic potential (1.69 folds; MCF-7: 0.049±0.0074 mg/ml) were higher than that of cancer cell clusters with more metastatic potential (MDA-MB-231: 0.029 ±0.0015 mg/ml). These results affirm a higher metabolite consumption and semi-quantitative production of glutamate by 2D MCF-7 cell cultures.

The CI_{PBS} level readouts were evaluated with the present biosensing system using a range of glutamate concentration levels that included concentrations detected from MCF-7 and MDA-MB-231 clusters (0.02 mg/ml, 0.04 mg/ml, 0.06 mg/ml, 0.08 mg/ml, and 0.1 mg/ml) (FIG. 3A). CI level readouts were modified for parallel comparison of all concentrations, with the CI level obtained from PBS buffer standards defined as 1. FIG. 3B shows that the glutamate concentration of 0.02 mg/ml generated the most significant CI_{PBS} level (3.6±0.03), which was within the range of glutamate levels detected from cancer cell clusters with more metastatic potential (MDA-MB-231). Higher concentrations of glutamate (≥0.04 mg/ml) led to lower CI_{PBS} levels. Specifically, the lowest CI_{PBS} level was reported with the highest glutamate concentration tested (0.1 mg/ml; CI_{PBS} level: 1.1±0.04).

FIG. 3C shows that glutamate degradation by glutamate dehydrogenase could reduce CI PBS level readouts with the present biosensing system to levels corresponding to samples from cancer cell clusters of higher metastatic potential, thus confirming the role of glutamate as a chemorepellent. Besides, other chemoattractants such as volatile compounds could still be present in cancer samples, which retained the chemotactic preference of C. elegans towards cancer samples over healthy controls, despite the degradation of glutamate.

The detection limit of the present biosensing system was evaluated with samples from both metastatic and less-metastatic cancer cell clusters under various dilutions (10⁻¹, 10⁻², and 10⁻³). The threshold was determined by the false positive rate (8.71 ±3.33%) obtained from the proportion of C. elegans distributed to chambers loaded with samples from control groups. Chemotactic preference of C. elegans towards samples from metastatic cancer cell clusters was abolished at dilutions higher than 10−1 (FIG. 3D). The dilution factor of 10⁻² corresponded to an approximate glutamate concentration of 0.003 mg/ml.

### Example 3

### Determination of Biosensor'S Sensitivity and Specificity to Metastatic Breast Cancer Clinical Samples in Terms of Chemotactic Difference of C. elegans Among Various Cancer Samples

To determine the biosensor's sensitivity and specificity to metastatic cancer sample over non-metastatic cancer sample, the chemotactic difference of C. elegans between metastatic and non-metastatic samples obtained from urines of breast cancer patients was investigated. The chemotactic preference of C. elegans to a cancer sample in the biosensor was quantified by chemotaxis index (CI) relative to a non-metastatic sample, where the CI was defined by the following Equations:

$${CI}_{MCF} {-}_{7} = \frac{percentage of C . elegans attracted to sample 1}{percentage of C . elegans attracted to MCF - 7 standard} \qquad ( 2 )$$

To define the CI of metastatic cancer sample according to the above Equations, a standard derived from a non-metastatic breast tumor cell line MCF-7 grown in Dulbecco's modified Eagle's medium (DMEM) (Gibco, USA) supplemented with 10% FBS (Gibco, Waltham, MA, USA), 1% penicillin-Streptomycin (Gibco) and maintained in T25 flasks (Biofil, Guangzhou, China) at 37° C. with 5% CO₂ was prepared. After reaching 80% -90% confluency, cells were harvested and passaged with 2.3×10⁵ cells per milliliter in 24 well plates, and every well added 300 μl cell suspension. After 48 hours of growth, the supernatant was collected as non-metastatic breast cancer (MCF-7) standards. Also, a pure PBS was used as a negative control.

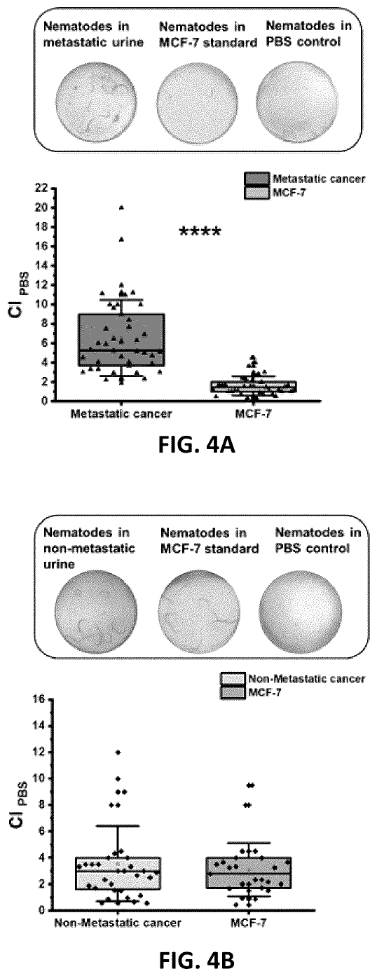

When the metastatic breast cancer urine sample, MCF-7 standards, and PBS control were applied to the present biosensing system, it was found that C. elegans were significantly attracted by the metastatic breast cancer urine sample. C. elegans had a significantly higher chemotactic preference to metastatic breast cancer patients' urine samples over MCF-7 standard (FIG. 4A). There was no significant difference between non-metastatic breast cancer patients' urine samples and the MCF-7 standard (FIG. 4B). As expected, the MCF-7 standard was more attractive to C. elegans when compared with healthy individual urine samples (FIG. 4C). Moreover, it was found that the CI_{MCF-7} of metastatic breast cancer urine samples was above 2; CI_{MCF-7} of non-metastatic breast cancer urine samples were below 2; and CI_{MCF-7} of healthy individual urine samples were below 1 (FIGS. 4D and 4E). Therefore, based on the CI_{MCF-7} distribution of different cohorts, a threshold of 2 was set for stratifying their metastatic potential. In other words, cancer patients with a CI_{MCF-7} of urine sample greater than 2 were regarded as having metastatic potential, and cancer patients with a CI_{MCF-7} of urine sample greater than 1 but smaller than 2 were regarded as having no risk of metastasis temporarily (FIG. 4E). Based on the above metastasis definition in terms of CI_{MCF-7}, the present biosensing system could result in high specificity (88.2%) and sensitivity (93.3%) towards metastatic breast cancer urine samples, which was more sensitive than gold standards (e.g., CT and MRI) and with a satisfactory specificity comparable to those obtained by gold standards.

Test samples in various pH values were also applied to the biosensing system to evaluate the effect of pH on C. elegans. It was found that C. elegans avoid samples at pH values greater than 10, and based on this result, the pH values of all the urine samples before applied to the biosensor were measured and adjusted to be within a range of 5.5 to 10.

### Example 4

### Validation of Biosensor's Potential to Detect Circulating Tumor Cells

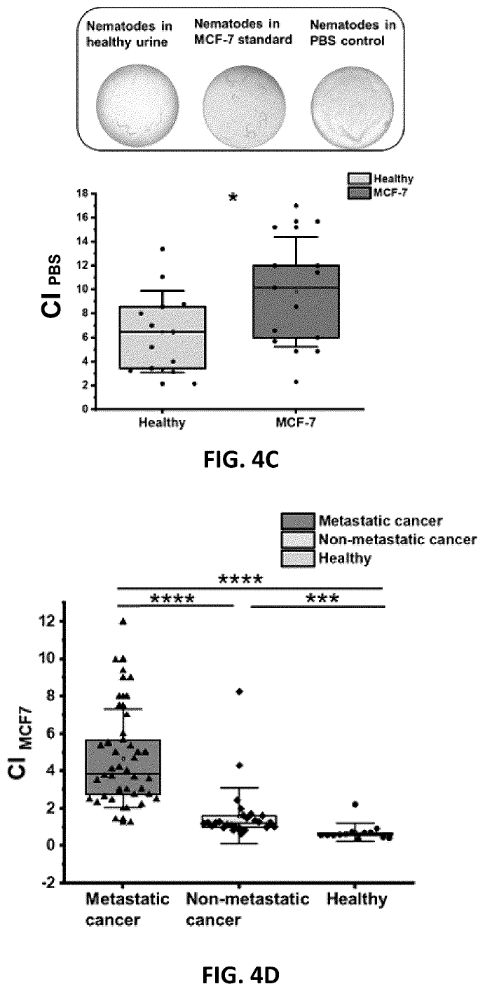

To determine whether the present biosensing system could be an effective tool for screening circulating tumor cells (CTCs) from blood samples, samples of single metastatic breast cancer cells, MDAMB-231 (ATCC Cat #HTB-26, RRID:CVCL 0062) spiked in the white blood cells (WBC) were applied to the biosensor to mimic a metastatic cancer blood sample in order to observe the response of C. elegans. It was found that C. elegans were attracted by MDAMB-231 cells (FIG. 5A), where those not spiked in WBC attracted more than those spiked in WBC, and the attractance increased gradually with an increasing number of MDAMB-231cells (FIG. 5B). The attractance did not disappear when MDAMB-231 cells mixed with WBC (FIG. 5C). From these results, it is suggested that C. elegans has the potential to detect and screen CTCs in blood samples when applied to the present biosensing system, apart from its chemotactic preference to metastatic cancer urine samples.

### Example 5

### Profiling Metabolic-Based Analytes for Stratification of Breast Cancer Patients by Raman Spectroscopy

To determine whether an analyte is a potential chemoattractant or chemorepellant for nematodes, Raman spectroscopy was utilized to obtain the spectra of urine samples from breast cancer patients with and without metastasis loaded onto silicon wafer substrates. Wavelet denoising and baseline correction techniques were employed in this example to process the representative Raman spectra of the urine samples. Two representative metabolic analytes, urea and L-proline, in breast cancer urine samples were studied.

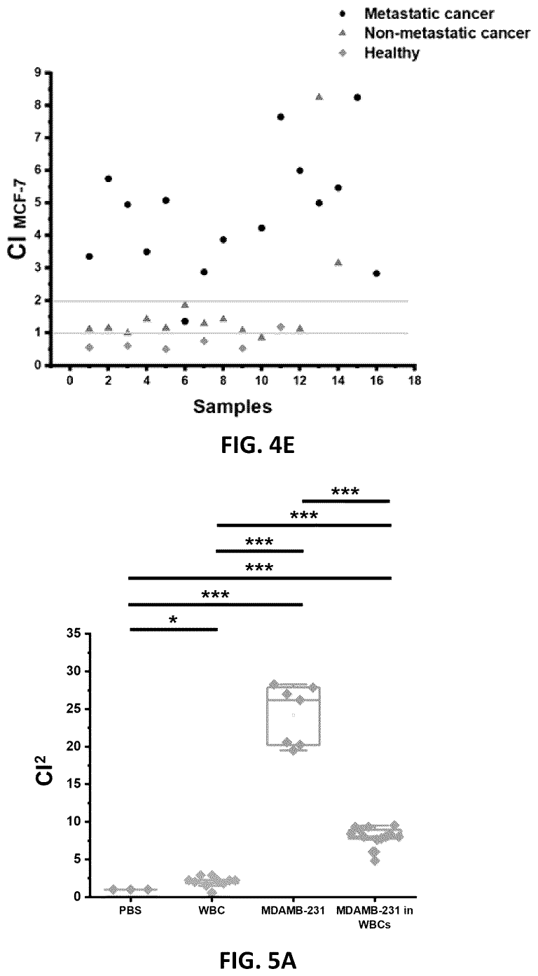

As seen from FIG. 6A, the processed Raman spectra were similar between metastatic and non-metastatic cancer patients' urine samples, but there were significant differences in five peaks, namely 856, 914, 944, 1008, and 1048 cm⁻¹. The Raman intensity of urine from non-metastatic patients was significantly higher than that of metastatic patients' urine at the peaks of 856, 914, 1008, and 1048 cm⁻¹ corresponding to urea and L-proline, respectively, demonstrating that certain metabolic analytes such as urea and L-proline analyzed in this example could be used to distinguish between these two cohorts.

When compared with healthy individuals, urine-derived metabolic compounds (glucose, urea, pyroglutamate, lactate, trisodium citrate, lactic acid, glutamic acid, citric acid, glutamine and gamma-aminobutyric acid) were found to decrease in relative concentration with cancer phenotype based on Raman spectra, gene expression profiles and liquid chromatography-mass spectrometry. Urine contains urea, chloride, sodium, potassium, sulfate, phosphate, other minor ions and proteins, and water. Twelve analytes (urea, L-proline, glucose, trisodium citrate, lactic acid, glutamic acid, CaCl₂, MgSO₄, KPO₄, citric acid, glutamine, Gamma-aminobutyric acid, and L-pyroglutamic acid) were known to be potential urinary marker. Therefore, two of the twelve metabolic analytes were selected at this time to prepare standard analyte solutions and measure their Raman intensities in order to identify and quantify their analyte frequency in urine samples of patients with and without metastasis (FIGS. 6B and 6C).

As seen from FIG. 6B, the Raman spectrum of urea corrected with a baseline of 40 mg/mL urea solution shows one significant prominent peak at 1008 cm⁻¹ that was assigned by an in-plane bending vibration of C—N bond in urea. From FIG. 6C, the Raman spectrum of L-proline corrected with a baseline of 1000 mg/mL L-proline solution shows three prominent peaks observed at 856, 914, and 1048 cm⁻¹, in which the first two correspond to symmetric stretching vibration of C—C bond, C—N bond in the pyrrolidine ring of L-proline while the third corresponds to an in-plane bending vibration of C—H bond in the pyrrolidine ring of L-proline. Although Raman spectra of urea and L-proline in solid state had significantly more distinctive peaks than that measured in their liquid state, the most significant Raman intensity peaks remained distinct in the Raman spectra obtained from liquid state of the analytes, i.e., 1008, 856, 914 and 1048 cm⁻¹ peaks are still clearly visible in the processed Raman spectrum of urea and L-proline, indicating the potential origins of the two peaks assignments. The Raman intensities of these peaks differed between metastatic and non-metastatic samples, suggesting that urea and L-proline could be used to stratify metastatic patients from non-metastatic patients.

To quantify the target analytes in clinical urine samples, calibration curves were plotted from Raman intensities measured from the prepared standard solutions of urea and L-proline, respectively, with varying concentrations. The corresponding calibration curves are shown in FIGS. 6D and 6E, respectively, for urea and L-proline standard solutions.

To avoid interference from the background, the Raman intensities of standards were calibrated by subtracting the peak intensity corresponding to water. The regression formula for urea was determined using the four-parameter (4-PL) fitting model as Equation (3):

$$y ( 2.5 - 40 mg / mL ) = \frac{176.667 + 0.299}{1 + {( \frac{x}{9.945} )}^{- 2.72}} - 0.299 ( {R}^{2} = 0.9991 ) \qquad ( 3 )$$

where x is urea concentration, y is Raman intensity, and R² is the coefficient of determination.

Similarly, the regression equation for L-proline was determined using the linear regression model as Equation (4):

y(72.5-1000 mg/mL)=−6.252+0.09x (R²=0.9883) (4)

where x is L-proline concentration, y is Raman intensity, and R² is the coefficient of determination.

The established urea and L-proline calibration curves (FIGS. 6D and 6E) could be used to quantify the corresponding analyte concentrations in clinical urine samples.

### Example 6

### Estimating Metastatic Risk Based on CI and Profiles of Urea and L-Proline

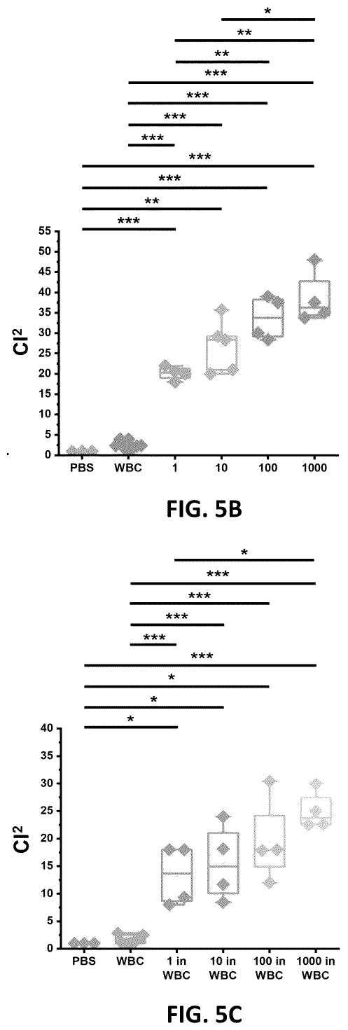

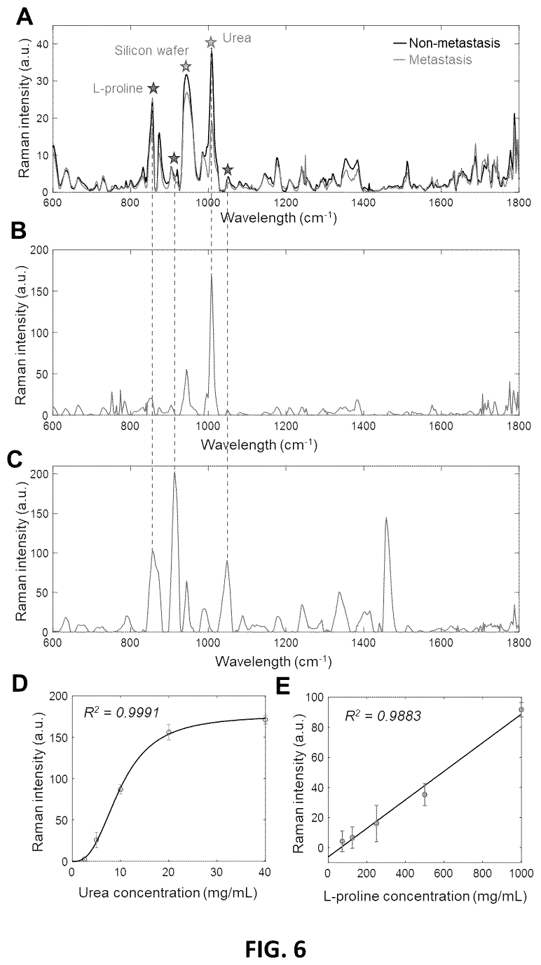

As Example 5 has suggested that Raman spectroscopy can easily determine the presence of urea and L-proline in clinical urea samples, and using different best-fitting models to determine calibration curves of urea and L-proline can quantify the concentration of the urea and L-proline in the clinical urine samples, profile of these two metabolic analytes in metastatic breast cancer urine sample and that in the non-metastatic breast cancer urine sample can be generated. Since lower levels of L-proline had been found in the urine of mice in the metastatic gastric cancer group than non-metastatic gastric cancer group, and proline catabolism was found to be higher in metastases than in primary breast cancers, and also the urea levels had also been found to have a moderate correlation with cancer progression, there appears to be a correlation between the variation of urea and L-proline levels in urine and the metastasis potential in a breast cancer sample. To verify, the profile of urea and L-proline in metastatic and non-metastatic breast cancer urine samples needs to be generated. FIGS. 7A and 7B demonstrate that non-metastatic urine samples had 2.2-fold higher urea concentrations and 2.3-fold higher L-proline concentrations than those in the metastatic samples. Similar profile of urea and L-proline in terms of Raman intensity measured at their corresponding distinct peaks in metastatic and non-metastatic urine samples has been observed (FIG. 8).

To determine a clinically relevant range of urea and L-proline concentrations and corresponding nematode chemotaxis scores at various urea and L-proline concentrations, heat maps reflecting the corresponding nematodes' preference to various concentrations of these two metabolic analytes were generated, which are shown in FIGS. 7C and 7D, respectively. It is suggested that the score was higher (around 6) at lower urea and L-proline concentrations, which correspond to metastatic cancer.

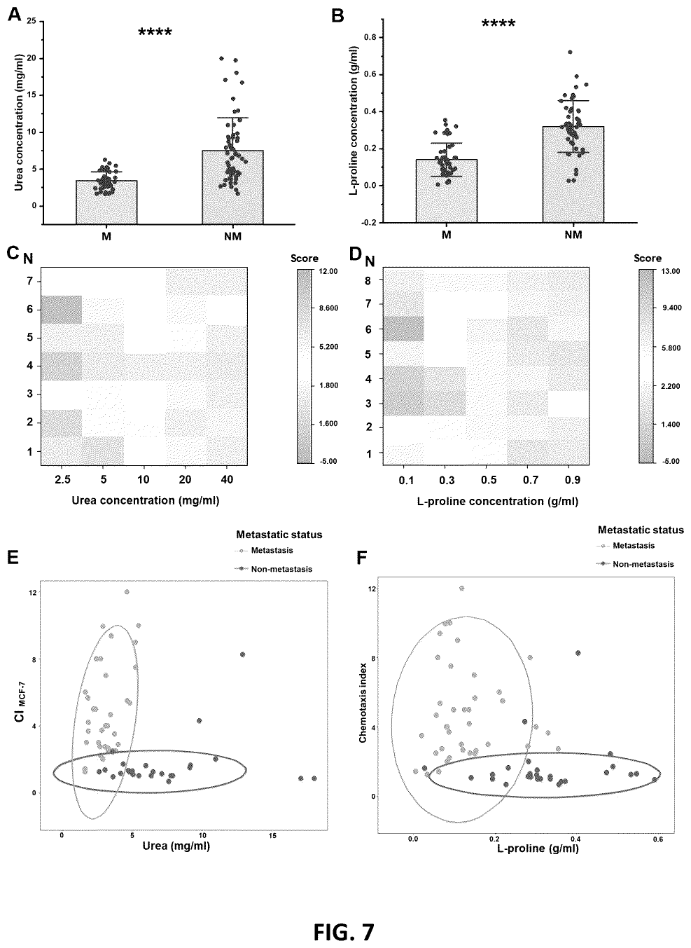

The correlation between CI_{MCF-7} and urea/L-proline concentrations in urine samples was then investigated. The higher the concentrations of urea and L-proline were, the lower was the CI_{MCF-7}, indicating non-metastatic status (FIGS. 7E and 7F). A mixture of both metabolites with the average level in clinical samples was applied to see if there is an attractive synergistic effect on nematodes because the mixture of both metabolites attracted nematodes (FIG. 9). It is demonstrated that a mixture of urea and L-proline had a higher score (around 10±1.35) with levels in metastatic samples (FIG. 9) than a single substance (only urea or L-proline), indicating an attractive synergistic effect of urea and L-proline.

Overall, it is suggested that low concentrations of urea and L-proline in metastatic urine samples had a higher level of chemoattraction to nematodes, indicating that urea and L-proline were biological cues that enabled nematodes to distinguish metastatic urine samples from non-metastatic ones, and are potential metastasis risk biomarkers.

### Example 7

### Personalized Biosensing Assay Kit for Remote Testing

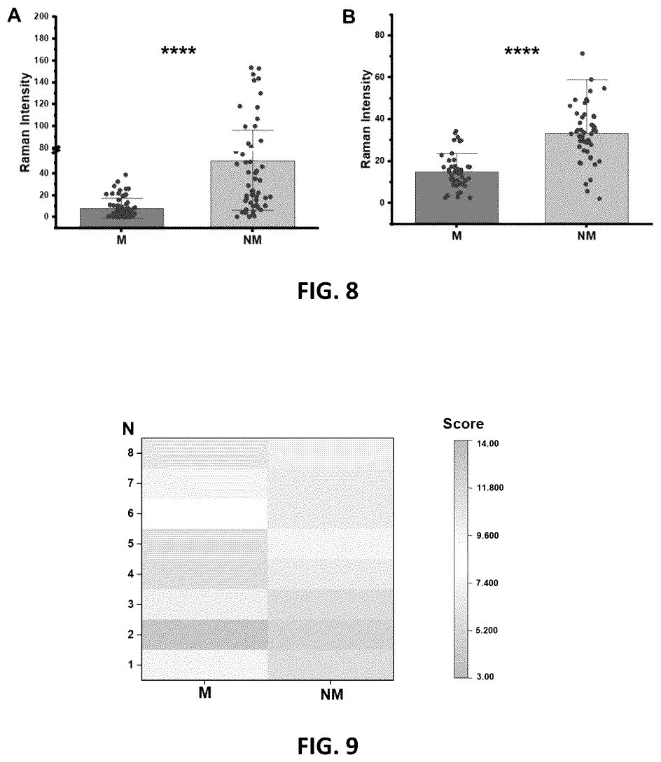

A smartphone-based integrated platform for visualizing and quantifying nematode distribution directly on the biosensor in situ was provided to realize home-based testing and analysis. A personalized kit for such integrated platform comprised the biosensor depicted in Example 1 and FIG. 1B, a biosensor holder, Z stage, and droplet lens for use with the smartphone (FIG. 10A). A PDMS-based droplet lens component was designed to be placed on a smartphone's rear-facing camera, allowing for quick and easy transformation into a portable microscope for visualizing the biosensing assay results. The direct biosensing assay could be processed on the go with other components.

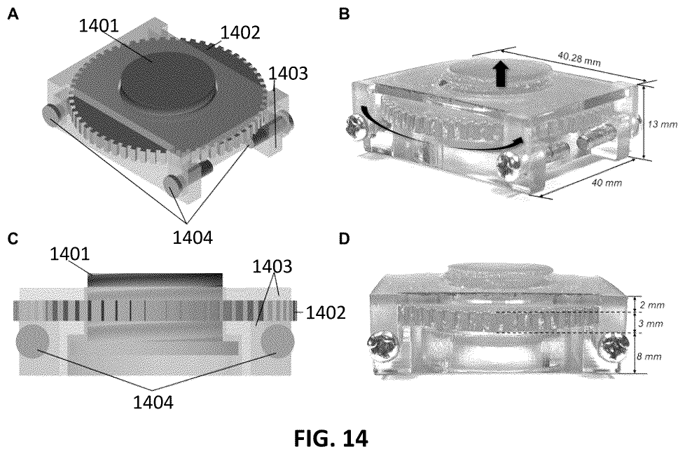

The Z stage was included in the kit for adjusting the focusing distance between the droplet lens and the bio-incubation chambers, an example of which is shown in FIG. 14. As seen from FIGS. 14A and 14C, a pair of clamps 1403 held the stage rotor 1402 in the required Z plane. The shaft 1401 could be adjusted by spinning the stage rotor 1402 anti-clockwise to obtain a focused view of the bio -incubation chambers. The biosensor could be rotated to view each chamber because it was mounted on the chip holder in a fixed XY position. Users can observe nematodes in each chamber in situ using a smartphone imaging detection module to capture images of the bio-incubation chambers through the window of the first layer of the biosensor designed for microscopy detection (FIG. 10B).

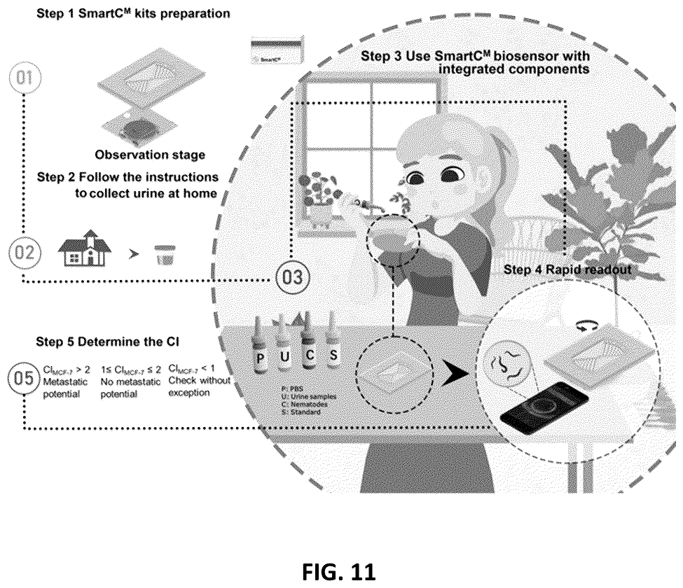

To verify the feasibility of household testing, the viability of nematodes at different time points was validated. FIG. 12A shows that the nematodes' viability remained above 80% after 24 hours. To investigate whether other elements in urine may affect the accuracy of the biosensing assay result, urine protein, urine glucose, and urine occult blood were studied, the results suggest that urine protein did not affect CI results for metastasis detection. In certain embodiments, basic urine tests such as urine test strips may be performed before the biosensing assay using the present biosensing system to eliminate the influence of other elements such as glucose and occult blood in the urine.

To minimize the impact of diet and hydration on patients, it is recommended that users adhere to a set of procedures when collecting urine samples, including: (1) Washing hands before collecting the sample; (2) Collecting a mid-stream urine sample to prevent contamination with bacteria; (3) Consuming a light meal the day before sample collection; (4) Avoiding heavy exercise and drinking water prior to the assay; (5) Fasting before sample collection; (6) Taking note of any medications being taken.

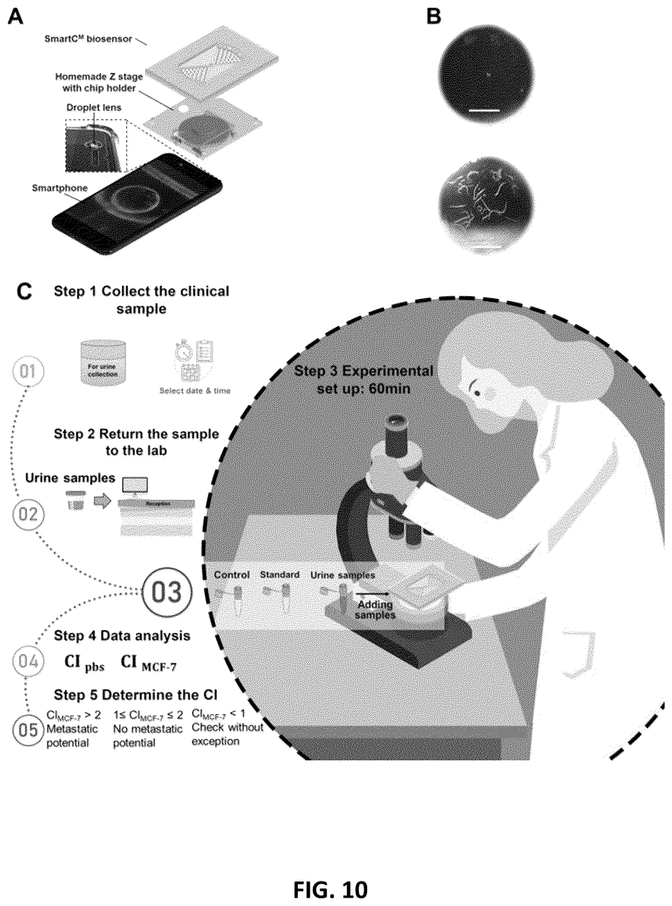

Overall, the non-invasive, low-cost integrated biosensing system complements existing diagnostic tests by providing a portable solution for assessing metastatic status using low volumes of urine samples, facilitating remote testing such as in rural regions or home-based routine screening (FIGS. 10C and 11), allowing patients at high risk of metastasis to seek help and initiate a timely intervention for treatment.

Although the invention has been described in terms of certain embodiments, other embodiments apparent to those of ordinary skill in the art are also within the scope of this invention. Accordingly, the scope of the invention is intended to be defined only by the claims which follow.

What is claimed is:

1. A biosensing system comprising a microfluidics-based biosensor, the biosensor comprising:
a first layer comprising at least a plurality of fluid inlets for loading one or more samples and a window; and
a second layer being disposed under the first layer and comprising an organism loading region and a plurality of bio-incubation chambers each having a plurality of obstructing mechanisms so that a type of organisms incubated therein is selectively attracted or repelled by one or more analytes in order to move away from the bio-incubation chambers towards the one or more analytes or stay within the bio-incubation chambers,
wherein a presence of the one or more analytes in the one or more samples indicates a metastatic status of the samples or a subject from which the samples are obtained.

2. The biosensing system of claim 1, wherein each of the plurality of fluid inlets is disposed adjacent to an edge of the window.

3. The biosensing system of claim 1, wherein the window is disposed substantially at a center of the first layer.

4. The biosensing system of claim 1, wherein each of the plurality of bio-incubation chambers is arranged in fan-like shape diverging substantially from a center of the second layer from a plan view of the biosensor.

5. The biosensing system of claim 1, wherein the obstructing mechanisms in each of the plurality of bio-incubation chambers are a plurality of polygonal structures.

6. The biosensing system of claim 5, wherein the plurality of polygonal structures is a plurality of triangular prism-shaped structures.

7. The biosensing system of claim 5, wherein each of the plurality of polygonal structures is evenly spaced apart from the other to define a plurality of runways for the type of organisms to move away from the bio-incubation chambers towards the one or more analytes.

8. The biosensing system of claim 1, wherein the organism loading region is disposed at an intersection of different bio-incubation chambers.

9. The biosensing system of claim 1, wherein each of the plurality of bio-incubation chambers has a cavity to accommodate the type of organisms and allow for a culture medium to incubate with the organisms.

10. The biosensing system of claim 1, wherein the type of organisms is a kind of nematodes.

11. The biosensing system of claim 10, wherein the type of nematodes is Caenorhabditis elegans.

12. The biosensing system of claim 1, wherein the first layer communicates with the second layer through one or more fluid channels, where each of the fluid channels or each intersection of two or more of the fluid channels has a larger width but a smaller height from a cross-sectional view than those of fluid channels within the bio-incubation chambers.

13. The biosensing system of claim 1, wherein the one or more samples comprise biological fluids, body fluids, metabolites, extracts from tissues or lesions, or media containing any of the above.

14. The biosensing system of claim 1, wherein the one or more samples are liquid biopsies comprising urine, saliva, mucus secretion, and extracts from tissues or lesions.

15. The biosensing system of claim 1, wherein the subject is human or non-human animal.

16. The biosensing system of claim 1, wherein the one or more analytes comprise urea, urine-derived metabolites and biological cells.

17. The biosensing system of claim 1, further comprising a holder of the biosensor, a linear translation stage, and a lens disposed under the biosensor, wherein the holder is attached to the linear translation stage for securing the biosensor on the linear translation stage.

18. The biosensing system of claim 17, wherein the linear translation stage is a precision z-axis translation stage with fixed x- and y-axis positions.

19. The biosensing system of claim 17, wherein the lens is a droplet lens or microlens for magnifying an area of interest in images of the biosensor captured by a smartphone or mobile device integrated with an imaging module

20. A method for differentiating a metastatic sample from a non-metastatic sample comprising providing the samples to the biosensing system of claim 1, wherein the biosensor of the biosensing system is loaded with a type of organisms that will be attracted or repelled by one or more analytes in the metastatic sample.

21. The method of claim 20, further comprising observing any movement of the organisms from where they are loaded towards a direction where the samples are loaded from the first layer and transported to the second layer of the biosensing system and quantifying the movement in terms of a percentage of the moving organisms towards where the samples are loaded and transported relative to a percentage of the moving organisms towards where a standard is loaded and transported in order to determine a chemotaxis index (CI) of the organisms to a particular sample relative to the percentage of the moving organisms towards the standard, wherein the standard is derived from a non-metastatic cell line medium, and wherein the sample is considered metastatic if the CI is greater than 2; otherwise, the sample is considered non-metastatic if the CI is between 1 and 2.

22. The method of claim 20, further comprising generating a Raman intensity profile for one or more of the analytes from a control in terms of Raman intensities measured at one or more peaks distinctive to the one or more analytes and comparing the Raman spectrum of the sample with the Raman intensity profile generated from the control in order to validate the metastatic status of the sample, wherein the control is prepared with a known concentration of the one or more analytes.

23. A kit for determining metastasis of a biological sample comprising the biosensing system of claim 1 and optionally one or more additional components comprising a biosensor holder, a precision z-axis translation stage, and a lens for magnifying an area of interest in images of the biosensor captured by a smartphone or mobile device integrated with an imaging module or by an imaging device.

24. The kit of claim 23, wherein the precision z-axis translation stage comprises a stage shaft, a rotor, a plurality of clamps and screws.
